(19) United States
(12) Patent Application Publication
(10) Pub. No.: US 2003/0138873 A1
(43) Pub. Date: Jul. 24, 2003

(54) Tissue engineered cartilage for drug discovery

(76) Inventors: Koichi Masuda, Wilmette, IL (US); Eugene J-M.A. Thonar, Lockport, IL (US); Brian Pfister, Wilmette, IL (US); Michael Hejna, Riverside, IL (US)

Correspondence Address: FOLEY & LARDNER, 150 EAST GILMAN STREET, P.O. BOX 1497, MADISON, WI, 53701-1497, US

(21) Appl. No.: 10/054,710

(22) Filed: Jan. 22, 2002

(51) Int. Cl. C12Q 1/22; C12Q 1/18

(52) U.S. Cl. 435/31; 435/32

(57) ABSTRACT

A culture system and method for determining the effect of a test agent on the development, homeostasis or degradation of engineered cartilage tissue. The engineered cartilage tissue is obtained by isolating chondrogenic cells and culturing them to obtain chondrocytes in a cell-associated matrix. The chondrocytes and cell associated matrix are then cultured on a semipermeable membrane to provide the engineered cartilage tissue. The engineered tissue, or one of its precursors, can be contacted with the test agent to determine what effect, if any, the test agent has on engineered cartilage.

### STATEMENT REGARDING FEDERALLY SPONSORED RESEARCH

This invention was made with Government support under grant No. 2-P50AR39239 awarded by the National Institutes of Health, National Institute of Arthritis, Musculoskeletal and Skin Diseases and grant No. AG04736 awarded by the National Institute on Aging. The Government has certain rights in this invention.

### FIELD OF INVENTION

The present invention relates to systems and methods utilizing engineered cartilage tissues. More particularly this invention relates to systems and methods utilizing engineered cartilage tissue to determine the effect compounds have on the cartilage matrix.

### BACKGROUND OF THE INVENTION

Articular cartilage is a complex avascular tissue made up of chondrocyte cells surrounded by extra-cellular matrix, which is composed mainly of collagens type II, IX, XI, proteoglycans, matrix protein and water. Although chondrocytes make up less than five percent of articular cartilage, they are responsible for producing and maintaining the extra-cellular matrix and thus proper joint function. As there is no blood supply to the cartilage matrix, cartilage has a limited ability to heal once damaged: not surprisingly it often undergoes progressive degenerative pathological changes. Effectively treating cartilage injuries is further complicated because a complete understanding of the mechanisms and natural history of cartilage injuries and the healing and regeneration of injured cartilage is lacking.

This lack of knowledge has both large human and economic costs because cartilage damage affects millions of people every year in the U.S. alone. Several hundred thousand people suffer injuries to articular cartilage in major joints, mainly due to sports injuries. It is also estimated that 50 million Americans suffer from osteoarthritis, a painful and debilitating disease that attacks the cartilage in joints.

Several different culture systems have been developed in an attempt to establish an in vitro cartilage model. Cartilage explant cultures are considered the closest and most relevant to in vivo cartilage but the intra- and intervariability of the results are often unacceptably large. Cartilage explants are also undesirable because it is difficult to obtain large amounts of human cartilage tissue and researchers must follow special procedures in order to comply with ethical research requirements. Artificial systems have been attempted using chondrocytes cultured in agarose or alginate gel media that promote the retention of the chondrocytic phenotype. However, a disadvantage of most chondrocyte culture systems is that they do not yield a tissue that resembles the cartilage matrix.

Thus, there continues to be a need for a culture system that accurately models cartilage tissue for use in therapeutic studies.

### SUMMARY OF THE INVENTION

In one embodiment of the present invention a method for determining the effect of a test agent on a tissue engineered cartilage matrix is described. According to this method an engineered cartilage tissue is produced by culturing isolated chondrogenic cells for an amount of time effective for allowing formation of a chondrogenic cell-associated matrix and culturing the chondrogenic cells with the cell-associated matrix on a semipermeable membrane in the presence of a growth factor for a time effective for allowing formation of the engineered cartilage tissue. One or more test agents are contacted with one or more cells or tissues selected from the group consisting of (a) the isolated chondrogenic cells prior to (i), (b) the chondrogenic cells during (i), (c) the chondrogenic cells and cell-associated matrix prior to (ii), (d) the chondrogenic cells and cell-associated matrix during (ii), and (e) the engineered cartilage tissue. The effect the one or more test agents has on the contacted cells or tissue is also measured in this method. The method can be carried out in the presence or absence of a known modulator of cartilage tissue.

The composition of the chondrogenic cell-associated matrix can vary, and can include aggrecan, collagen types II, IX and XI, and hyaluronan. The tissue can also be made of at least about 5 mg/cc³ aggrecan, with the ratio of aggrecan to hyaluronan ranging from about 10:1 to about 200:1, and the ratio of aggrecan to collagen ranging from about 1:1 to about 10:1.

Chondrogenic cells can be isolated from various sources, such as articular cartilage and fibrocartilages. More specifically the cells can be isolated from costal cartilage, nasal cartilage, auricular cartilage, tracheal cartilage, epiglottic cartilage, thyroid cartilage, arytenoid cartilage or cricoid cartilage. Exemplary fibrocartilages include tendon, ligament, meniscus or intervertebral disc.

Based on these test one or more test agents can be identified that have desirable properties and can be produced as a therapeutic drug. Kits for carrying out these methods are also provided by the present invention. These kits can include instructions for carrying out these methods, one or more reagents useful in carrying out these methods, one or more enzymes capable of degrading the engineered cartilage tissue, and/or a dye or antibody capable of labeling a component of the engineered cartilage tissue.

Objects and advantages of the present invention will become more readily apparent from the following detailed description.

### BRIEF DESCRIPTION OF THE DRAWINGS

FIG. 1 is a graph depicting the effect of IL-1α on proteoglycan turnover in engineered cartilage tissue; and

FIG. 2 is a bar graph showing the effect of IL-1α on proteoglycan content in engineered cartilage tissue.

### DETAILED DESCRIPTION OF PREFERRED EMBODIMENTS

One embodiment of the present invention provides methods of testing the effects of different test agents on engineered cartilage tissue. In the present methods, engineered cartilage tissue is exposed to one or more compounds (test agents) to determine what effect, if any, these compounds have on the growth, homeostatic balance and/or degradation of the cartilage tissue. The present culture system and methods are also useful for studying the anabolic and catabolic processes that are in balance in matrix homeostasis. In one method, engineered cartilage tissue is cultured to effectively mimic the physical properties and chemical and biological constituents of articular cartilage. Preferred methods for culturing engineered cartilage can be found in U.S. Pat. No. 6,197,061 entitled “In Vitro Production of Transplantable Cartilage Tissue, Cohesive Cartilage Produced Thereby, and Method for the Surgical Repair of Cartilage Damage” issued to Masuda et al., the contents of which are explicitly incorporated herein.

Generally, chondrogenic cells are isolated and cultured to produce chondrocytes with a chondrogenic cell-associated matrix. The chondrocytes and their cell-associated matrix are then cultured on a semi-permeable membrane in the presence of one or more growth factors to produce an engineered cartilage tissue.

Isolation of Chondrocytes/Chondrogenic Cells

Chondrogenic cells useful in the present methods can be isolated from essentially any tissue containing chondrogenic cells. As used herein, the term “chondrogenic cell” is understood to mean any cell which, when exposed to appropriate stimuli, can differentiate into a cell capable of producing and secreting components characteristic of cartilage tissue. The chondrogenic cells can be isolated directly from pre-existing cartilage tissue, for example, hyaline cartilage, elastic cartilage, or fibrocartilage. Specifically, chondrogenic cells can be isolated from articular cartilage (from either weight-bearing or non-weight-bearing joints), costal cartilage, nasal cartilage, auricular cartilage, tracheal cartilage, epiglottic cartilage, thyroid cartilage, arytenoid cartilage, cricoid cartilage, tendon, ligament, meniscus and intervertebral discs, either nucleus pulposus or annulus fibrosus. Tendon and ligament cells can also be isolated from a specific source, such as the anterior cruciate ligament or Achilles tendon.

Alternatively, chondrogenic cells can be isolated from bone marrow. See for example, U.S. Pat. Nos. 5,197,985 and 4,642,120, and Wakitani et al. (1994) J. bone Joint Surg. 76:579-591, the disclosures of which are incorporated by reference herein. Chondrogenic cells can also be derived from stem cells.

Suitable chondrocytes can be isolated from any suitable mammalian source organism, including, without limitation, human, orangutan, monkey, chimpanzee, dog, cat, rat, mouse, horse, cow, pig, and the like. Chondrocytes can be either isolated from sources having normal cartilage or cartilage which is known to be defective in some manner, such as having a genetic defect.

Chondrocyte cells used for preparation of the in vitro cell culture device of the present invention can be isolated by any suitable method. Various starting materials and methods for chondrocyte isolation are known (see generally, Freshney, Culture of Animal Cells: A Manual of Basic Techniques, 2d ed., A. R. Liss Inc., New York, pp 137-168 (1987); Klagsburn, “Large Scale Preparation of Chondrocytes,” Methods Enzymol. 58:560-564 (1979).

If the starting material is a tissue in which chondrocytes are essentially the only cell type present, e.g., articular cartilage, the cells can be obtained directly by conventional collagenase digestion and tissue culture methods. Alternatively, the cells can be isolated from other cell types present in the starting material. One known method for chondrocyte isolation includes differential adhesion to plastic tissue culture vessels. In a second method, antibodies that bind to chondrocyte cell surface markers can be coated on tissue culture plates and then used selectively to bind chondrocytes from a heterogeneous cell population. In a third method, fluorescence activated cell sorting (FACS) using chondrocyte-specific antibodies is used to isolate chondrocytes. In a fourth method, chondrocytes are isolated on the basis of their buoyant density, by centrifugation through a density gradient such as Ficoll.

It can be desirable in certain circumstance to utilize chondrocyte stem cells rather than differentiated chondrocytes. Examples of tissues from which stem cells for differentiation, or differentiated cells suitable for transdifferentiation, can be isolated include placenta, umbilical cord, bone marrow, skin, muscle, periosteum, or perichondrium. Cells can be isolated from these tissues through an explant culture and/or enzymatic digestion of surrounding matrix using conventional methods.

Culture in Medium for the Production of Chondrocyte Cell-Associated Matrix

Isolated chondrocytes/chondrogenic cells are suspended at a density of preferably at least about 10⁴ cells/ml in an appropriate medium, such as agarose or sodium alginate. The cells are cultured under conditions effective for maintaining their spherical conformation conducive to the production, upon the chondrocyte membrane, of a cell-associated matrix similar to that found in vivo. Preferably, chondrocytes are cultured in alginate for at least about five days to allow for formation of a cell-associated matrix. The media within which the chondrocytes are cultured can contain a stimulatory agent, such as fetal bovine serum, to enhance the production of the cell-associated matrix.

In an alternative aspect of the invention, the culture medium for the chondrocytes can further include exogenously added specific growth factors. The addition of specific growth factors, for example those not already present in fetal bovine serum, such as osteogenic protein-1, can act as an effective stimulator of matrix formation. In this aspect of the invention, growth factor is added to the medium in an amount to near-maximally stimulate formation of the cell-associated matrix, which is dependent on the type of cells stimulated. In the case of BMP4 or OP-1, typically for chondrocytes, 50 ng to 200 ng/ml can be used. For ligament, tendon and meniscus cells, amounts up to 1 μg/ml can be used.

Preferably, amplification of chondrocytes or chondrogenic cells in the growth medium does not induce loss of the chondrocyte phenotype as occurs when amplification is performed in monolayer culture. In one example a chondrocyte phenotype is a phenotype typical in articular cartilage wherein the cells have (i) a spherical shape and the ability to synthesize and accumulate within the matrix significant amounts of (ii) aggrecan and (iii) type II collagen without (iv) accumulating within the matrix an effective amount of type I collagen. As used herein, a minimal amount of collagen type I means less than about 10% of all collagen molecules that become incorporated within the matrix. Chondrocytes cultured in alginate retain their spherical shape (typical of chondrocytes) and maintain a large amount of matrix.

A phenotypically stable articular cartilage chondrocyte can also retain the ability to effectively incorporate the major macromolecules into a cartilage-like matrix. Normal articular chondrocytes can express small amounts of mRNA for collagen type I that they do not translate. Further, articular chondrocytes cultured in alginate beads for several months can synthesize some collagen type I molecules, but the latter never become incorporated into the forming matrix. Consequently, the appearance of small amounts of newly-synthesized collagen type I molecules in the medium does not necessarily denote the onset of dedifferentiation. Further, hyaluronan is not a marker of the chondrocytic phenotype since it is synthesized in large amounts by many other cell types. However, it is an essential constituent of the cartilage matrix.

Cells that are phenotypically stable should synthesize at least about 10 times more aggrecan than collagen (on a mass basis). Further, the ratio of aggrecan to hyaluronan in the matrix produced by articular chondrocytes can remain above about 10.

Chondrocyte with Cell-Associated Matrix

Culture of chondrocytes in alginate results in the production of an extracellular matrix (ECM) that is organized into two compartments: (i) a cell-associated matrix compartment that metabolically resembles the pericellular and territorial matrices of native tissues, and (ii) a further removed matrix compartment that metabolically resembles the interterritorial matrix of native tissue.

The formation of a highly structured cell-associated matrix around each chondrocyte is desired for several reasons. First, the cell-associated matrix is anchored to the cell via receptors such as anchorin CII (which binds to collagen)and CD44 (which binds to hyaluronan in proteoglycan aggregates). Once this matrix has been reestablished, the cells are much less likely to become dedifferentiated. Second, the chondrocyte turns over proteoglycan aggrecan and thus remodels this matrix relatively rapidly. The chondrocyte is much less effective in remodeling the further removed matrix.

Preferably, the cell-associated matrix compartment of the ECM produced during culture in alginate includes aggrecan (the major cartilage proteoglycan), collagen types II, IX and XI, and hyaluronan. Aggrecan molecules in the cell-associated matrix are formed principally as aggregates bound to receptors (including CD44) on the chondrocyte cell membrane via hyaluronan molecules.

The relative proportions of each component in the cell-associated matrix vary depending on the length of time in culture. Preferably, the cell-associated matrix has at least about 5 mg/cc³ of aggrecan, a ratio of aggrecan to hyaluronan (mg/mg)between 10:1 and 200:1, and a ratio of aggrecan to collagen (mg/mg) from 1:1 to about 10:1. Further, the molecular composition of the cell-associated matrix (around each cell) and further removed matrix (between the cells) can be altered by specific modifications of the culture conditions. These modifications involve the physical arrangement of the culture system and application of various growth factors. Manipulation of matrix production and organization are central to the engineering of articular cartilage in vitro for surgical treatment of cartilage injury.

Preferably, the contents of collagen and of the pyridinoline crosslinks of collagen increase with time of culture. The crosslinks in particular show a dramatic increase in concentration after two weeks of culture. By keeping the length of the culture period relatively short, the collagen fibrils in the cell-associated matrix do not become overly crosslinked. A tissue that has good functional properties but is relatively deficient in crosslinks is easier to manipulate. Tissues with higher amounts of crosslinking can be desired when more mature cartilage is sought to be simulated.

In another embodiment of the present invention, the chondrocytes are isolated from fibrocartilage, either white or yellow (elastic). These chondrocytes retain their fibrocartilage phenotype thus producing a cell-associated matrix having collagen and proteoglycan contents more characteristic of the fibrocartilage source from which they were isolated. In this embodiment, type I collagen can be the predominant collagen type depending upon the tissue desired to be replicated.

Recovery of Chondrocytes with Their Cell-Associated Matrix

Preferably, recovery of chondrocytes with their cell-associated matrix is accomplished by solubilizing alginate beads after a sufficient culture period. Alginate beads are first solubilized using known techniques. The resulting cell suspension then is centrifuged, separating the cells with their cell-associated matrix in the pellet from the components of the further removed matrix in the supernatant.

Culturing the Chondrocyte with Their Cell-Associated Matrix on a Semipermeable Membrane.

In this aspect of the invention, the chondrocytes with their cell-associated matrix isolated as described above, are further cultured on a semipermeable membrane. Preferably, a cell culture insert is placed into a plastic support frame and culture medium flows around the cell culture insert. In this aspect, the cell culture insert includes a semipermeable membrane. The semipermeable membrane allows medium to flow into the cell culture insert in an amount effective for completely immersing the chondrocytes and their cell-associated matrix.

Preferably, the semipermeable membrane allows the chondrocytes to have continuous access to nutrients while allowing the diffusion of waste products from the vicinity of the cells. In this aspect, the membrane should have a pore size effective to prevent migration of chondrocytes through the pores and subsequent anchoring to the membrane, preferably not be more than about 5 microns. Further, the membrane utilized should have a pore density effective for providing the membrane with sufficient strength so that it can be removed from its culture frame without curling, and with sufficient strength such that the tissue on the membrane can be manipulated and cut to its desired size. Preferably the membrane should have a pore density of at least about 8×10⁵ pores/cm². The membrane can be made of any material suitable for use in culture. Examples of suitable membrane systems include but are not restricted to: (i) Falcon Cell Culture Insert [Polyethylene terephthalate (PET) membrane, pore size 0.4 to 3 microns, diameter 12 to 25 mm]; (ii) Coaster Transwell Plate [Polycarbonate membranes, pore size, 0.1 to 5.0 microns, diameter 12 to 24.5 mm]; (iii) Nunc Tissue Culture Insert (Polycarbonate Membrane Insert: pore size, 0.4 to 3.0 microns, diameter 10 mm to 25 mm); Millicell Culture Plate Insert [PTFE (polytetrafluoroethylene) membrane, polycarbonate, pore size 0.4 to 3.0 microns, diameter 27 mm].

The beads containing chondrocytic cells are cultured in a growth medium, such as equal parts of Dulbecco's modified Eagle medium and Ham's F12 medium containing 20% fetal bovine serum (Hyclone, Logan, Utah), about 251 g/ml ascorbate and antibiotic, such as 50 μg/ml gentamicin (Gibco). In an alternative approach, the beads are cultured in a closed chamber that allows for continuous pumping of medium. Preferably, the medium contains fetal bovine serum containing endogenous insulin-like growth factor-1 at a concentration of at least about 10 ng/ml. In this usage, fetal bovine serum can also be considered a growth factor. Several serum free culture media such as HL-1™, PC-1™ and UltraCulture™ (BioWhittaker) can be used in place of fetal bovine serum. Suitable growth factors that can be exogenously added to the medium to maximally stimulate formation of the cell-associated matrix include but are not limited to osteogenic protein-1 (OP-1), bone morphogenic protein-2 and other bone morphogenetic proteins, cartilage-derived morphogenetic protein, platelet-derived growth factor, fibroblast growth factor, transforming growth factor beta, and insulin-like growth factor.

In another aspect of the invention, cells with their reestablished cell-associated matrix are further cultured in medium on the semipermeable membrane for an amount of time effective for allowing formation of a cohesive cartilage matrix. Culture times will generally be at least about 3 days under standard culture conditions. Partial inhibition of matrix maturation prior to implantation is important in providing a matrix that is not as stiff as mature cartilage, but which has enough tensile strength to retain its shape and structure during handling.

Mechanical properties of the cartilage matrix can be controlled by increasing or decreasing the amount of time that the cartilage tissue is cultured on the membrane. Longer culture time will result in increased crosslink densities.

Cartilage Matrix

Preferably, the cartilage matrix that forms on the semipermeable membrane has a concentration of aggrecan of at least about 5 mg/cc³ and the cartilage matrix contains an amount of hyaluronan effective for allowing all the newly synthesized molecules to become incorporated into proteoglycan aggregates. The matrix of the tissue formed on the membrane contains aggregated aggrecan molecule at a concentration not less than 5 mg/cc³, a ratio of aggrecan to hyaluronan of about 10:1 to about 200:1, and a ratio of aggrecan to collagen of about 1:1 to about 10:1. The engineered cartilage used in the present methods closely resembles naturally occurring articular cartilage in its physicochemical properties in a short period of time, typically about 14 days. It is also preferable to remove the engineered cartilage from the semipermeable membrane.

The engineered cartilage tissue is used in a culture system to determine the effect of a test agent, alone or in combination with other agents, on the physical and chemical make-up of the engineered cartilage. As used herein, the term “test agent” is defined as a chemical compound that has no known modulating effect on the cartilage tissue at the stage of cartilage development in which the test agent is administered. Accordingly, one skilled in the art will understand that the term “test agent” is dependent on multiple factors including at least the compound to be tested and the developmental stage of the cartilage in which the compound is tested. Thus the same compound may be a test agent for one stage of cartilage development, such as culturing of chondrogenic cells to produce a cell-associated matrix, but not be a test agent for another stage of cartilage development because the compound has a known effect on that stage of cartilage development.

In the culture system, the engineered cartilage is contacted with a test agent. The test agent can be applied to the culture system in the presence or absence of known modulators that directly act on the cartilage tissue, including matrix metalloproteinases (MMPs) and serine proteases, or of modulators that induce or inhibit these directly acting compounds, such as tumor necrosis factor-α (TNF-α) and cytokines such as interleukin-1 (IL-1), and modulators which act even further downstream in the process, such as retinoic acid (which regulates cytokine signaling) and lipopolysaccharide. Likewise, the test agent can be applied to the culture system in the presence of one or more additional test agents to determine the effect the combination of agents has on the engineered cartilage. In a preferred embodiment, the test agent is not a known modulator of cartilage tissue, such as IL-1 and growth factors. Thus, the test agent can be a compound that acts (i) directly on the cartilage tissue, (ii) on a compound that acts directly on the cartilage tissue, or (iii) on a modulator of a compound which acts directly on the cartilage tissue.

Similarly, the test agent can be applied to the culture system at one or more of the various stages of the engineered cartilage stage of development discussed above. For example, the test agent can be contacted with the isolated cells prior to, during or after: (i) chondrogenic cell culturing in growth medium to produce cell-associated matrix; (ii) recovery of the chondrocytes and the cell-associated matrix; (iii) culturing of the chondrocytes and cell-associated matrix on the semipermeable membrane; or (iv) removal of the engineered cartilage matrix from the semipermeable membrane. In this manner, the test agent can be examined for activity in any or all of: (i) maintaining chondrogenic cell viability; (ii) maintaining chondrogenic cell phenotype; (iii) modulation of growth of the cell-associated matrix; (iv) modulation of cartilage matrix production and growth; (v) modulation of cartilage homeostasis or (vi) modulation of cartilage degradation.

Preferably, a control experiment is run for comparison so that the effect of the test agent can be more readily evaluated. Typically the control experiment will exclude the test agent, one or more of the combination of test agents, one or more compounds that act directly on the cartilage tissue or one or more modulators of compounds that act directly on the cartilage.

Surprisingly and unexpectedly, it has been discovered that the engineered cartilage matrix discussed herein can be rapidly degraded, losing roughly half of its proteoglycan content within a single day after treatment with IL-1. Without limiting the scope of the invention, it is believed that the engineered tissue has a greater sensitivity to cytokines. This rapid degradation lends itself to high throughput screening methods because testing of compounds can be completed in a relatively short amount of time. Likewise, large amounts of engineered cartilage tissue can be quickly obtained and small samples can be removed therefrom for sampling in multi-well plates, thus leading to easy automation of the instant screening methods. Because several tissue samples can be obtained from the same piece of engineered tissue, intraassay and interassay variability of data generated according to the present methods is very low. Significantly, the present methods can be economically achieved because the engineered cartilage can be cultured and tested in the same well of a multi-well plate, thus requiring little manipulation or perturbation of the tissue. Another significant advantage of the present method results from the fact that testing can be completed without the addition of extrinsic radioactivity, which is typically accomplished through the use of radioisotope labeling.

When non-radioactive techniques are used to quantify the proportion of engineered cartilage components that are retained in the engineered tissue after treatment with the test agent or modulators of the metabolism of cartilage tissue or its metabolism, it is preferable to digest the engineered cartilage matrix enzymatically. Enzymatic digestion of the cartilage matrix can be achieved using one or more proteases, such as papain, chymopapain, pronase and proteinase K. After enzymatic digestion, the proteoglycan content and DNA content of the cartilage can be measured using several well-known techniques in the art, such as the DMMB method and Hoechst 33258-dye method, respectively. Additionally, the content of hydroxyproline (a measure of collagen), and the content of hyaluronan can be measured using reverse-phase HPLC and ELISA, respectively.

An additional advantage of the present invention is that the engineered cartilage tissue disclosed herein has homogeneous characteristics because the cartilage cells are initially obtained through the digestion of whole cartilage tissue. As is known in the art, cartilage is not a homogeneous tissue, but is instead made up of different layers. Aydelotte, M. B., R. R. Greenhill, et al. (1988). “Differences between sub-populations of cultured bovine articular chondrocytes. II. Proteoglycan metabolism.” Connect Tissue Res. 18(3): 223-34. Aydelotte, M. B. and K. E. Kuettner (1988). “Differences between sub-populations of cultured bovine articular chondrocytes. I. Morphology and cartilage matrix production.” Connect Tissue Res. 18(3): 205-22.] Thus, engineered tissue produced through other techniques can have widely disparate properties depending upon the technique used to obtain the cells, the amount of cells which are taken from each layer and the amount of cell dedifferentiation that occurs in producing the tissue. In contrast, the present tissue provides a more consistent cell population and ratio and thus enhanced reproducibility of experiments can be achieved. Likewise, more meaningful comparisons can be made between experiments because of the increase homogeneity of the engineered cartilage used herein.

The present culture system can also be used to mimic different pathological states in cartilage tissue, including physical injury and disease states, such as rheumatoid arthritis. According to this embodiment cartilage is cultured and then either artificially injured, such as by physically cutting or tearing the engineered cartilage tissue, or treated with factors, such as inflammatory mediators and cartilage matrix degrading compounds, known to cause the progression of disease states. The engineered cartilage mimicking a pathological state can then be treated with one or more test agents as described above to determine the effect the test agent has on the pathological state. In this embodiment, as in others, it may be desirable to isolate chondrogenic cells that are known to have a certain defect, such as a genetic defect.

After a test agent is identified as having a desired property, such as upregulating the production of cartilage, inhibiting cartilage degradation or enhancing cartilage degradation, the test agent can be identified and then either isolated or chemically synthesized to produce a therapeutic drug. Thus, the present methods can be used to make drug products useful for the therapeutic treatment of cartilage tissues in vitro and in vivo.

The present invention also provides kits for carrying out the methods described herein. In one embodiment, the kit is made up of instructions for carrying out any of the methods described herein. The instructions can be provided in any intelligible form through a tangible medium, such as printed on paper, computer readable media, or the like. The present kits can also include one or more reagents, buffers, culture media, culture media supplements, enzymes capable of degrading the engineered cartilage, antibodies for labeling a specific component of the cartilage tissue, chromatic or fluorescent dyes for staining or labeling a specific component of the cartilage tissue, radioactive isotopes for labeling specific components of the engineered cartilage, and/or disposable lab equipment, such as multi-well plates in order to readily facilitate implementation of the present methods. Components of the cartilage tissue to be stained or labeled can include a fragment of the matrix cleaved by enzymatic action, which may or may not be released into the surrounding media.

This invention is further illustrated by the following non-limiting examples.

### EXAMPLES

### Example 1

### Cartilage Matrix Turnover Measured Using Radiolabelling

Engineered Cartilage Tissue Formation: Engineered cartilage tissue was prepared as described in U.S. Pat. No. 6,197,061. Briefly, bovine articular chondrocytes were isolated from steer (14-18 months) and cultured in beads of 1.2% low viscosity alginate (Keltone LV, Kelco) at 4 million cells/ml in daily changes of medium containing 20% FBS, 25 μg/ml ascorbate and 10 μg/ml gentamicin, as described above in Mok, et al. On day 7, beads were dissolved by incubation for 20 minutes in 55 mM NaCitrate in 150 mM NaCl. The cells with their CM (cellular matrix) were recovered by mild centrifugation. The cells with their CM were resuspended in complete medium and seeded onto a tissue culture insert (0.4 μm pore size; 23 mm diameter, Falcon). After 7 and 21 days in culture, the de novo formed tissue was separated from the porous membrane and 4 mm diameter discs were punched out using a skin biopsy punch.

Discs of engineered cartilage tissue were punched out from engineered cartilage tissue on day 7 and 21 and were incubated for 16 h in complete medium containing ³⁵S-sulfate (20 μCi/ml). After washing to remove unincorporated radioisotope, the discs were cultured for 7 days in isotope-free medium with or without IL-1α (1 ng/ml). The medium in all cases was changed and collected for the measurement of ³⁵S-labeled proteoglycans (³⁵S-PGs). At various times, discs were collected and the content of ³⁵S-PGs in each disc and the corresponding spent medium fraction was measured by a rapid filtration assay (4). For each set of conditions (i.e. with or without IL-1α) the amount of radiolabeled PGs remaining in each matrix pool was plotted against time of chase to measure the average half-life of ³⁵S-PGs in each compartment. The data were fitted to the double exponential decay equation: y=ae^{-bx}+ce^{-d}+e and the half-life calculated based on b and d. Mok, et al., Aggrecan synthesized by mature bovine chondrocytes suspended in alginate: Identification of two distinct metabolic matrix pool, Biol. Chem., 269, 33021-33027 (1994)

### Example 2

### Cartilage Matrix Turnover Measured Without Radiolabelling

Discs of engineered cartilage tissue was punched out from engineered cartilage tissue on day 7 and cultured for 5 days in complete medium with/without IL-1α (1 ng/ml). Engineered cartilage tissue was obtained as described above. On days 1, 2, 3 and 5, the discs were harvested and digested with papain. After digestion the contents of sulfated PG and DNA were measured by the DMMB method and Hoechst 33258-dye method, respectively [5].

Statistical Analysis: Statistical analysis was performed comparing IL-1-treated and untreated samples by one-way ANOVA, using the Fisher's PLSD test as a post hoc test.

Results from Examples 1 and 2

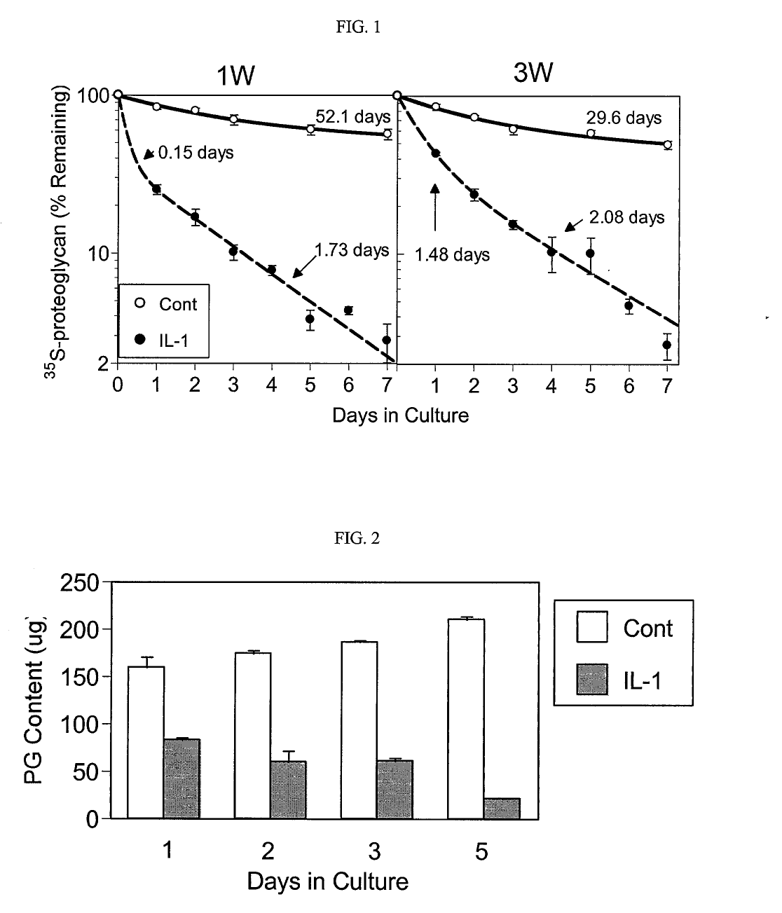

The half-life of ³⁵S-PGs synthesized on days 7 and 21 was slightly longer than the half-life previously reported for ³⁵S-PGs in cartilage explants of bovine animals of the same age (FIG. 1). The addition of IL-1 to the medium caused a rapid increase in the appearance in the medium of ³⁵S-PGs, representing mostly proteolytically degraded fragments of aggrecan. The loss of ³⁵S-PGs in the tissue was slightly more pronounced in tissue radiolabeled on day 7 than in tissue radiolabeled on day 21 in culture (T½=1.73 days [1W], 2.08 days [3W]). Further, ³⁵S-PGs radiolabeled at the earlier time point exhibited a more clearly bimodal rate of disappearance from the tissue (T½=0.15 days [1W], 1.48 days [3W]).

Surprisingly and unexpectedly, measurement of PGs remaining in the engineered cartilage tissue at various times after treatment with IL-1 at 1 ng/ml (FIG. 2) demonstrated that quantification of the IL-1-induced promotion of PG loss from the matrix does not require radioisotopes, as it does in cartilage explants, especially where data is desired to be obtained rapidly. The present invention does not require the use of extrinsic radioactivity because the matrix structure provided in the present culture system can provide a cell-associated matrix which is looser, or less densely packed, so that the cartilage fragments can migrate out of the matrix. Cartilage explants, on the other hand, trap enzymatically degraded cartilage fragments due to its density. Even a single day of treatment with IL-1 at this concentration resulted in the loss of 50% of the PGs. Accordingly, characterization of the cartilage matrix, and thus the effect of test agents thereon, is simplified.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” “more than” and the like include the number recited and refer to ranges which can be subsequently broken down into subranges as discussed above. In the same manner, all ratios disclosed herein also include all subratios falling within the broader ratio.

One skilled in the art will also readily recognize that where members are grouped together in a common manner, such as in a Markush group, the present invention encompasses not only the entire group listed as a whole, but each member of the group individually and all possible subgroups of the main group. Accordingly, for all purposes, the present invention encompasses not only the main group, but also the main group absent one or more of the group members. The present invention also envisages the explicit exclusion of one or more of any of the group members in the claimed invention.

All references disclosed herein are specifically incorporated herein by reference thereto.

While preferred embodiments have been illustrated and described, it should be understood that changes and modifications can be made therein in accordance with ordinary skill in the art without departing from the invention in its broader aspects as defined in the following claims.

What is claimed is:

1. A method for determining the effect of a test agent on a tissue engineered cartilage matrix, comprising:
(A) culturing an engineered cartilage tissue comprising the steps of:
(i) culturing isolated chondrogenic cells for an amount of time effective for allowing formation of a chondrogenic cell-associated matrix; and
(ii) culturing the chondrogenic cells with the cell-associated matrix on a semipermeable membrane in the presence of a growth factor for a time effective for allowing formation of the engineered cartilage tissue;
(B) contacting one or more test agents with one or more cells or tissues selected from the group consisting of (a) the isolated chondrogenic cells prior to (i), (b) the chondrogenic cells during (i), (c) the chondrogenic cells and cell-associated matrix prior to (ii), (d) the chondrogenic cells and cell-associated matrix during (ii), and (e) the engineered cartilage tissue; and
(C) measuring the effect the one or more test agents has on the contacted cells or tissue.

2. The method of claim 1 wherein the chondrogenic cell-associated matrix comprises aggrecan, collagen types II, IX and XI, matrix proteins and hyaluronan.

3. The method of claim 1 wherein the engineered cartilage tissue comprises collagen types II, IX and XI, hyaluronan and at least about 5 mg/cc³ aggrecan,
wherein the ratio of aggrecan to hyaluronan is about 10:1 to about 200:1, and the ratio of aggrecan to collagen is about 1:1 to about 10:1.

4. The method of claim 1 wherein the isolated chondrogenic cells are from articular cartilage.

5. The method of claim 1 wherein the isolated chondrogenic cells are from costal cartilage, nasal cartilage, auricular cartilage, tracheal cartilage, epiglottic cartilage, thyroid cartilage, arytenoid cartilage or cricoid cartilage.

6. The method of claim 1 wherein the isolated chondrogenic cells are from fibrocartilage.

7. The method of claim 6 wherein the fibrocartilage is ligament, tendon, meniscus or intervertebral disc.

8. The method of claim 1 wherein step (i) comprises culturing the chondrogenic cells on an alginate medium.

9. The method of claim 1 wherein step (C) comprises measuring the amount of proteoglycan in the engineered cartilage tissue.

10. The method of claim 1 wherein step (C) is performed without the addition of extrinsic radioactivity.

11. The method of claim 10 wherein step (C) comprises enzymatically degrading the engineered cartilage tissue.

12. The method of claim 11 wherein step (C) further comprises staining the enzymatically degraded engineered cartilage tissue with a dye.

13. The method of claim 1 wherein the engineered cartilage tissue is removed from the semipermeable membrane prior to being contacted with the test agent.

14. The method of claim 1 further comprising:
(D) identifying one or more test agents that have desirable properties; and
(E) producing the one or more test agents as a therapeutic drug.

15. A kit for determining the effect of a test agent on a tissue engineered cartilage matrix comprising instructions for carrying out the method of claim 1.

16. The kit of claim 15 further comprising one or more of:
(i) one or more reagents;
(ii) an enzyme capable of degrading the engineered cartilage tissue;
(iii) a dye capable of labeling a component of the engineered cartilage tissue; and
(iv) an antibody capable of labeling a component of the engineered cartilage tissue.

17. A method for determining the effect of a test agent on a tissue engineered cartilage matrix, comprising:
(A) culturing an engineered cartilage tissue comprising the steps of:
(i) culturing isolated chondrogenic cells for an amount of time effective for allowing formation of a chondrogenic cell-associated matrix; and
(ii) culturing the chondrogenic cells with the cell-associated matrix on a semipermeable membrane in the presence of a growth factor for a time effective for allowing formation of the engineered cartilage tissue;
(B) contacting one or more test agents with one or more cells or tissues selected from the group consisting of (a) the isolated chondrogenic cells prior to (i), (b) the chondrogenic cells during (i), (c) the chondrogenic cells and cell-associated matrix prior to (ii), (d) the chondrogenic cells and cell-associated matrix during (ii), and (e) the engineered cartilage tissue in the presence of a known modulator of cartilage tissue; and
(C) measuring the effect the one or more test agents has on the contacted cells or tissue.

18. The method of claim 17 wherein the chondrogenic cell-associated matrix comprises aggrecan, collagen types II, IX and XI, and hyaluronan.

19. The method of claim 17 wherein the isolated chondrogenic cells are from articular cartilage.

20. The method of claim 17 wherein the isolated chondrogenic cells are from costal cartilage, nasal cartilage, auricular cartilage, tracheal cartilage, epiglottic cartilage, thyroid cartilage, arytenoid cartilage or cricoid cartilage.

21. The method of claim 17 wherein the isolated chondrogenic cells are from fibrocartilage.

22. The method of claim 21 wherein the fibrocartilage is ligament, tendon, meniscus or intervertebral disc.

23. The method of claim 17 wherein step (i) comprises culturing the chondrogenic cells on an alginate medium.

24. The method of claim 17 wherein the engineered cartilage tissue comprises collagen types II, IX and XI, hyaluronan and at least about 5 mg/cc³ aggrecan, wherein the ratio of aggrecan to hyaluronan is about 10:1 to about 200:1, and the ratio of aggrecan to collagen is about 1:1 to about 10:1.

25. The method of claim 17 wherein step (C) comprises measuring the amount of proteoglycan in the engineered cartilage tissue.

26. The method of claim 17 wherein step (C) is performed without the addition of extrinsic radioactivity.

27. The method of claim 26 wherein step (C) comprises enzymatically degrading the engineered cartilage tissue.

28. The method of claim 27 wherein step (C) further comprises staining the enzymatically degraded engineered cartilage tissue with a dye.

29. The method of claim 17 wherein the modulator of the engineered cartilage tissue is a matrix stimulating agent, cytokine or TNF-α.

30. The method of claim 29 wherein the cytokine is interleukin-1.

31. A kit for determining the effect of a test agent on an engineered cartilage tissue comprising instructions for carrying out the method of claim 17.

32. The kit of claim 31 further comprising one or more of:
(i) one or more reagents;
(ii) an enzyme capable of degrading the engineered cartilage tissue;
(iii) a dye capable of labeling a component of the engineered cartilage tissue; and
(iv) an antibody capable of detecting a component of the engineered ivcartilage tissue.

33. The method of claim 17 further comprising:
(D) identifying one or more test agents that have desirable properties; and
(E) producing the one or more test agents as a therapeutic drug.

34. The method of claim 17 further comprising removing the engineered cartilage tissue from the semipermeable membrane prior to contacting the engineered cartilage tissue with the test agent.

35. The method of claim 17 wherein steps (A) and (B) occur in the same well of a multiwell plate.
